(19) United States
(12) Patent Application Publication
(10) Pub. No.: US 2017/0273569 A1
(43) Pub. Date: Sep. 28, 2017

(54) THERMOMETER DEVICE

(71) Applicants: JORDAN BARBER, JACKSON, MS (US); CHEVAN BAKER, KANSAS CITY, MO (US); FRED HARRIS, LAKE, MS (US); JANN BUTLER, MOSS POINT, MS (US); GORDON SKELTON, MADISON, MS (US)

(72) Inventors: JORDAN BARBER, JACKSON, MS (US); CHEVAN BAKER, KANSAS CITY, MO (US); FRED HARRIS, LAKE, MS (US); JANN BUTLER, MOSS POINT, MS (US); GORDON SKELTON, MADISON, MS (US)

(73) Assignee: Jackson State University, Jackson, MS (US)

(21) Appl. No.: 15/387,376

(22) Filed: Dec. 21, 2016

Related U.S. Application Data
(60) Provisional application No. 62/314,098, filed on Mar. 28, 2016.

(51) Int. Cl. A61B 5/01; A61B 5/00

(52) U.S. Cl. CPC A61B 5/01; A61B 5/6892; A61B 5/6807; A61B 5/6804

(57) ABSTRACT

A device for measuring the temperature of a lower extremity of a subject in need of such monitoring. The device is especially suited for monitoring the foot temperature of a subject with diabetes or another condition that could lead to lower blood circulation and temperature in the lower extremities of the subject. Methods of preventing complications from such diseases, disorders, syndromes, or conditions are provided. The past recorded temperatures of the subject may be stored for comparisons and trends analyses.

### CROSS-REFERENCE TO RELATED APPLICATIONS

This application claims priority to U.S. Provisional Application No. 62/314,098 to Jordan Barber et al. filed on Mar. 28, 2016, the contents of which are incorporated herein by reference in its entirety.

### FIELD OF THE INVENTION

The present invention is generally directed toward a device for measuring the temperature of an extremity of the anatomy of a subject, and more particularly to monitoring the foot temperature of a subject with diabetes or another condition causing relatively low blood circulation and temperature in the extremities of the subject.

### BACKGROUND OF THE INVENTION

Some diseases, disorders, syndromes, and conditions cause decreased blood flow to the extremities of the body. One such disease is diabetes mellitus. A diabetic patient has abnormal (high) glucose levels in the blood, affecting its flow to the lower extremities, such as the feet and toes. The decreased blood flow can lead to serious complications, including numbness, neuropathy, pain, and even tissue damage, ulceration, amputation, and worse. The Mississippi Department of Health said the Magnolia State ranked second in the nation for prevalence of diabetes in 2012. Accordingly, four of every 1,000 deaths in Mississippi resulted from complications of the disease.

There is a need for a means of monitoring the blood flow or other related symptoms of diabetic complications in a subject's lower extremities to prevent or lessen the damage to tissue in these subjects.

### SUMMARY OF THE INVENTION

The goal of the present invention is to address the shortcomings of the prior art and to provide a device for measuring the temperature directly (and blood flow indirectly) of a lower extremity of a subject in need thereof. Accordingly, in one aspect, the present invention provides a device for measuring the temperature of at least one foot of a subject in need thereof comprising: a structural platform, a plurality of temperature sensors, a power supply, and a control box. The structural platform may be a mat, pad, or wearable pad. The plurality of temperature sensors may be thermistor sensors. The power supply may be a battery. The control box further comprises a processor and a memory storage device. The control box may further comprise a transceiver.

In another aspect, the present invention provides a method of preventing tissue damage in a subject in need thereof comprising the steps of measuring the temperature of a lower extremity of the subject in need thereof with a plurality of temperature sensors as a first data set, processing the first data set collected from the plurality of temperature sensors, measuring the temperature of the lower extremity of the subject in need thereof with the plurality of temperature sensors as a second data set, processing the second data set collected from the plurality of temperature sensors, analyzing the first and second data sets, providing an instruction for a user or caregiver to take a remedial action based on the results of the analyzing step.

### BRIEF DESCRIPTION OF THE DRAWINGS

Further advantages of the invention will become apparent by reference to the detailed description of preferred embodiments when considered in conjunction with the drawings:

FIG. 1 is a top plan view of an exemplary embodiment of the temperature measuring device in the form of a mat.

FIGS. 2A & 2B depict an exemplary embodiment of the temperature measuring device in the form of an individual foot pad. FIG. 2A is a top plan view of the temperature measuring device pad integrated into an insole. FIG. 2B is a side elevation view of the temperature measuring device pad integrated into a hosiery article.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

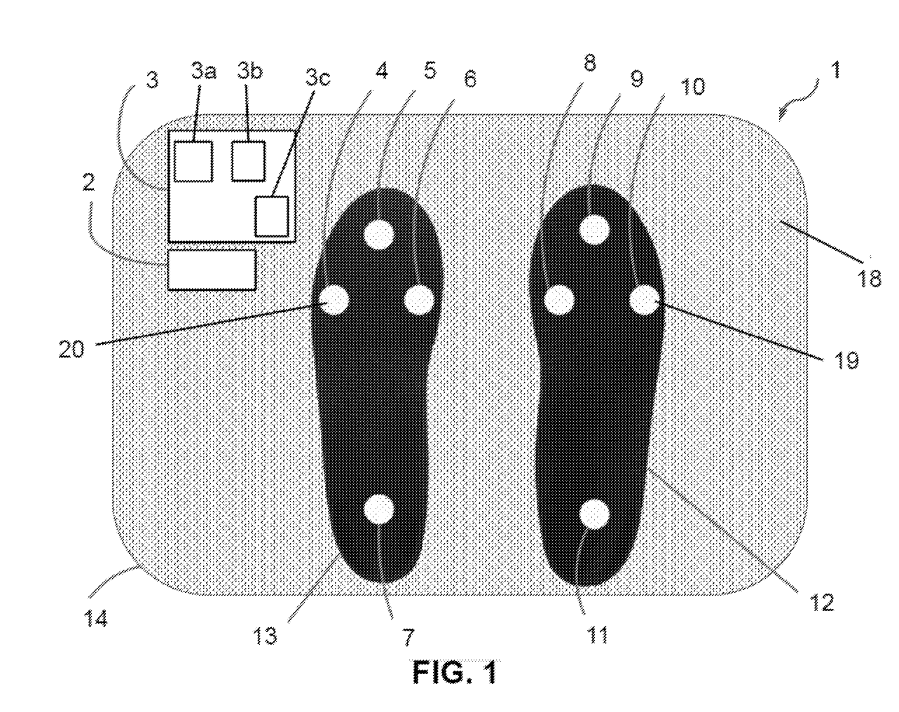

The present invention provides an electronic temperature measuring device 1 for measuring the temperature of an extremity of a subject in need thereof and taking appropriate remedial action based on the results of such measurement readings. A subject in need thereof should be understood in this context to be a person suffering from, suspected to be suffering from, and/or at risk of suffering from at least one disease, disorder, syndrome, or condition that causes at least some relative lowering of the body temperature in one or more extremities of the body, whether periodically or otherwise. Diabetes mellitus is one example of such an at least one disease, disorder, syndrome, or condition that causes at least some relative lowering of the body temperature in one or more extremities of the body. A preferred embodiment of the temperature measuring device 1 is configured to measure the temperature of the foot. Referring to the drawings, FIG. 1 illustrates an exemplary embodiment of a foot temperature measuring device 1 with a platform in the form of a foot mat 14. A foot mat 14 is a preferred form for a foot temperature measuring device 1 because it allows for easy measuring with a plurality of temperature sensors 4-11 while a subject is standing, which provides for an accurate temperature reading of the subject's foot/feet temperature. A particularly preferred embodiment includes the foot temperature measuring device 1 integrated within an anti-fatigue mat 14 like that shown in FIG. 1.

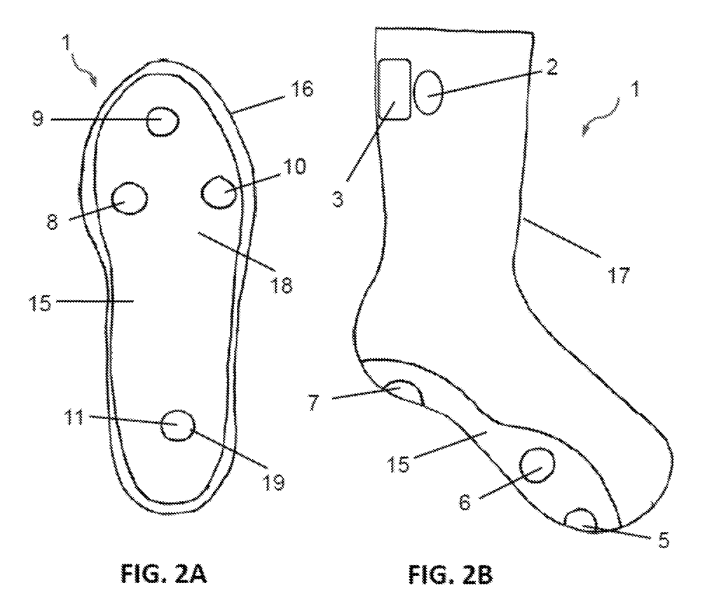

The foot temperature measuring device 1 can take many different advantageous forms, including, but not limited to, mats 14 and foot pads 15. Pad-type 15 foot temperature measuring devices 1 are particularly suited for integration into insoles 16 for shoes and other footwear, socks 17 or other forms of hosiery, and other articles. It should be understood that the selected form may result in a pair of separately operating foot temperature measuring devices 1, such as a pad 15 integrated into an insole 16 article (see FIG. 2A) or a pad 15 integrated into a hosiery 17 article (FIG. 2B). See below for further discussion of the operation of the foot temperature measuring device 1. The only limitation on form is that which will allow for the foot temperature measuring device 1 to take an accurate reading of the subject's body temperature at his or her foot/feet. Generally, the temperature sensors (e.g., 4 in FIG. 1) must be positioned and/or held within sufficient proximity to the subject's foot for a predetermined length of time in order to allow the foot temperature measuring device 1 to take an accurate reading. Thus, while a subject can satisfy the proximity and temporal duration requirements of the foot temperature measuring device 1 by standing on or placing their foot/feet on top of the foot temperature measuring device 1 while in a seated position, some subjects in need thereof may not be able to stand or sit up on their own or for very long comfortably. For these subjects, mat-type 14 or pad-type 15 foot temperature measuring devices 1 may be held against the foot/feet of the subject for at least the required temporal duration. A person of ordinary skill in the art will readily understand that the proximity and temporal duration requirements may be accomplished by manually holding or by employing straps (not shown) to hold the foot temperature measuring device(s) 1 to the bottom of the subject's foot/feet or by using a wearable 16, 17 pad-type 15 embodiment (see FIGS. 2A & 2B). A wearable foot temperature measuring devices 1 is also advantageous for monitoring temperature of the subject's foot/feet during times of sleeping. In all cases, the foot temperature measuring device 1 is comfortable to the user/subject in need thereof while using the foot temperature measuring device 1.

The foot temperature measuring device 1 has a plurality of temperature sensors 4-11 disposed on or near an instrument surface 18 of the foot temperature measuring device 1 such that the subject's temperature can be measured. Thermistor-type sensors 19 are particularly preferred due to their accuracy in a variety of environmental conditions. It has been found that the plurality of temperature sensors 4-11 are more accurate when kept free of moisture, which can be a problem wet feet from after a bath/shower or from perspiration. To overcome this issue, the plurality of temperature sensors 4-11 must be protected from moisture by a protective coating 20 over the instrument surface 18 of the foot temperature measuring device 1 and/or individual temperature sensors. In all forms, the plurality of temperature sensors 4-11 are substantially flat to provide the user/subject with a comfortable experience while standing on or wearing the foot temperature measuring device 1. The plurality of temperature sensors 4-11 may otherwise be a thermocouple, a resistance thermometer, or known other electrical temperature sensor such as infrared body thermometers, temperature strips, digital heat sensors, infrared wave sensors, and basal thermometers.

The placement of the plurality of temperature sensors 4-11 within the foot temperature measuring device 1 is configured to best achieve an accurate temperature gauge of the subject's foot/feet. Thus, the plurality of temperature sensors 4-11 are preferably placed at contact points of the foot, including the heel, ball, and toes (phalanges). As can be appreciated in FIG. 1, an exemplary embodiment of the foot temperature measuring device 1 is provided with a thermistor-type 19 temperature sensor 4 is disposed at the midfoot-ball of left foot closest to the fifth metatarsal/phalange area, a thermistor-type 19 temperature sensor 5 is disposed at the front-left foot toe/phalange area, a thermistor-type 19 temperature sensor 6 is disposed at the midfoot-ball of left foot first metatarsal/phalange area, a thermistor-type 19 temperature sensor 7 is disposed at the rear-left foot heel area, a thermistor-type 19 temperature sensor 8 is disposed at the midfoot-ball of right foot first metatarsal/phalange area, a thermistor-type 19 temperature sensor 9 is disposed at the front-right foot toe/phalange area, a thermistor-type 19 temperature sensor 10 is disposed at the midfoot-ball of right foot fifth metatarsal/phalange area, and a thermistor-type 19 temperature sensor 11 is disposed at the rear-right foot heel area. It should be understood that more or fewer temperature sensors may be employed as desired. The temperature sensors 4-11 may be attached directly to the instrument surface 19 or embedded within the foot temperature measuring device 1 below the instrument surface 18 such that the plurality of temperature sensors 4-11 will be able to register accurate temperature readings of the subject's foot/feet.

As briefly discussed above, a subject in need of temperature monitoring uses the foot temperature measuring device 1 by placing his or her foot/feet onto the instrument surface 18. As shown in FIG. 1, a mat-type 14 device 1 may be provided with a right foot placement guide 12 and a left foot placement guide 13 to assist the user/subject to properly place the foot/feet over the plurality of temperature sensors 4-11. Wearable forms, such as pad-type 15 devices 1 may be configured to be worn on a particular foot (i.e., left and right, with an appropriate foot placement guide 13/12) or universally (i.e., left or right). In order to obtain an accurate temperature reading, the subject's foot/feet must be positioned and/or held within sufficient proximity to the subject's foot/feet for a predetermined length of time. A sufficient proximity should be understood to mean touching or near touching (directly or through a thin layer of clothing, such as socks or hosiery) with or without pressure onto the plurality of temperature sensors 4-11. A person of ordinary skill in the art will be able to discern an appropriate sufficient proximity of the subject's foot/feet to the plurality of temperature sensors 4-11 through routine methods based on the form of the foot temperature measuring device 1 by testing accuracy of the readings. A predetermined length of time is any time period that is required or sufficient for the plurality of temperature sensors 4-11 to measure and record an accurate temperature. In some embodiments, the predetermined length of time is two minutes or less, such as 60 seconds to 120 seconds, 90 seconds to 120 seconds, or 60 seconds to 90 seconds. In preferred embodiments, the predetermined length of time one minute or less, such as 30 seconds to 60 seconds, 45 seconds to 60 seconds, or 30 seconds to 45 seconds. In more preferred embodiments, the predetermined length of time 30 seconds or less, such as 1 second to 30 seconds, 15 seconds to 30 seconds, 1 second to 15 seconds, 1 second to 5 seconds, 5 seconds to 10 seconds, or 10 seconds to 15 seconds. In particularly preferred embodiments, the predetermined length of time is 15 seconds.

The plurality of temperature sensors 4-11 are in electric communication with a power supply 2 and a control box 3. The power supply 2 can be any source of voltage to power the plurality of temperature sensors 4-11 and the control box 3, which is discussed in more detail below. In preferred embodiments, the power supply 2 is a battery, such as, for example, a 9V battery 2. The battery 2 may be rechargeable in some embodiments. The foot temperature measuring device 1 preferably is configured to save power in order to conserve battery life, when so provided. For example, the foot temperature measuring device 1 can be kept in an “off” or low power “sleep” mode under normal conditions. The foot temperature measuring device 1 may have an on/off switch that is accessible to a user/subject without the need for tools. Some embodiments may “wake up” periodically to an “on” condition at predetermined (programmed time periods stored within the memory 3a) in order to take a reading before returning to the “off” or “sleep” mode. Some embodiments may be configured to trigger an “on” condition when the user/subject provides a triggering amount of pressure (trigger pressure can be programmed and stored within the memory 3a) so that a reading measurement is only taken when the user/subject is well within the proximity sufficient for an accurate reading. Also, this latter embodiment may further include a predetermined time duration between data collection (time period can be programmed and stored within the memory 3a). For example, if the user/subject is standing on a mat-type 14 device 1 while performing some task, the device can be triggered to only make a reading every two minutes, five minutes, 10 minutes, 15 minutes, etc. In less preferred embodiments, the power supply 2 is a 120V alternating current, such as available in electric outlets.

The plurality of temperature sensors 4-11 is in electric communication with the control box 3. An output of an electronic signal carrying the temperature reading information is transferred from the plurality of temperature sensors 4-11 to the control box 3. The control box 3 processes (by processor 3b, microcontroller, CPU, or similar device well-known in the art) the electronic signal from the plurality of temperature sensors 4-11 to extrapolate the temperature reading from each of the plurality of temperature sensors 4-11. The temperature readings form the plurality of temperature sensors 4-11 are stored in a memory 3a that is in electronic communication with the processor 3b. Preferably, the temperature readings data are given a time and date stamp according to their time of creation, and this information is stored in association with the temperature readings data. The memory 3a can be accessed by the microcontroller 3b in order to transfer the data stored therein to another computing device. The method of transfer can be any known method within the field of medical devices. For example, the data can be recalled and transferred to a hardware device (not shown), such as a flash memory device (an SD card, a USB memory storage device, or the like). In some embodiments, the memory 3a is a removable memory storage device, such as a flash memory device (an SD card, a USB memory storage device, or the like). In preferred embodiments, the microcontroller 3b is in electronic communication with an electromagnetic transceiver 3c for wirelessly transferring the stored data. In some embodiments having an electromagnetic transceiver 3c, the memory 3a can be omitted for direct transmission and storage of the data on a separate computing device's memory. The foot temperature measuring device 1 may be integrated directly with a doctor's office or medical establishment (e.g., a hospital or nursing home) by an Internet connection or a direct network connection to transfer the captured data directly to the computing device(s) thereof for convenient analysis by caregivers. In a particularly preferred embodiment, the data is transferred to a handheld computing device (e.g., a smartphone or tablet) with an app for collecting, analyzing, and/or storing.

It has been found that a subject's lower extremity temperature correlates well with blood flow where a decrease in blood flow relates to a decrease in temperature of a subject's foot. For example, a drop of four degrees Fahrenheit from a baseline temperature in one or both feet of a subject may indicate a period of decreased blood flow that could cause tissue damage complications for the subject's lower extremities if not acted upon. A baseline temperature may be an average baseline temperature preprogrammed into memory 3a, or it may be a personalized baseline temperature that may be programmed into memory 3a for the user/subject using the device 1. Therefore, the temperature data captured can be used by a caregiver of the subject, such as a nurse or doctor, to monitor the health of the subject and/or prescribe preventive/remedial action or corrective action based on the analysis of the transferred data. The data captured can be processed and stored for each individual sensor of the plurality of temperature sensors 4-11. The captured data can also be processed to provide an averaged temp for all sensors for one or both feet of the subject. The data captured can be processed to glean trends in temperature and blood flow for each foot and/or for each sensor. A person of ordinary skill in the art will readily understand that more precise readings, time stamps, and increased number of data points (i.e., sensors 4-11) can provide more data for discerning trouble spots within a given lower extremity of a subject that need special attention for preventive/remedial action or corrective action. By monitoring the temperature of the lower extremities of a subject, a foot or a portion thereof that is experiencing periods of lower temperature and lower blood flow can be found before tissue damage and ulceration occurs. The foot temperature measuring device 1 acts as an early warning device and, thereby, provides for a prevention of complications of diabetes, low blood flow, and/or low temperature of a lower extremity. In addition to using the data captured to diagnose and take remedial action(s), the data can alert the user/subject in need thereof by performing an audible or visual alarm when the temperature reading of any foot or individual temperature sensor falls below the baseline temperature.

The foot temperature measuring device 1 is convenient to use and care for. The foot temperature measuring device 1 is light weight, mobile, and easily transportable. The foot temperature measuring device 1 is waterproof for cleaning. Mat-type 14 devices 1 can be cleaned by wiping away soiling. Wearable forms are also capable of cleaning with water. Wearable insole-type 16 devices 1 may be removed from shoes or other footwear for cleaning. Wearable sock/hosiery-type 17 devices 1 are hand-washable with waterproof sealing of all electronic components and/or removable electronic components.

Some embodiments may be configured to determine a body mass index of a user/subject with mass/weight scales built into a mat-type 14 device 1 when coupled to a data for the user's/subject's height data (which may be preprogrammed into the memory 3a). Some embodiments may also have a percent body fat measurement capability. Some embodiments may be configured to integrate with other health apps, platforms, and systems. Alternative embodiments include providing a plurality of temperature sensors 4-11 for placement on a flat surface or directly on the user's/subject's skin surface or sock/hosiery article of the foot bottom.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

We claim:

1. An electronic temperature measuring device comprising: a plurality of temperature sensors, a power supply, and a processor.

2. The electronic temperature measuring device of claim 1, further comprising a structural platform.

3. The electronic temperature measuring device of claim 2, wherein the structural platform is selected from the group consisting of a mat, a pad, or a wearable pad.

4. The electronic temperature measuring device of claim 3, further comprising a protective coating that covers an instrument surface.

5. The electronic temperature measuring device of claim 3, wherein the mat is an anti-fatigue mat.

6. The electronic temperature measuring device of claim 3, wherein the pad is integrated into a wearable article selected from the group consisting of footwear insole and hosiery article.

7. The electronic temperature measuring device of claim 1, further comprising a memory storage device.

8. The electronic temperature measuring device of claim 1, further comprising a transceiver.

9. The electronic temperature measuring device of claim 1, wherein the plurality of temperature sensors are selected from the group consisting of thermistor sensors, thermocouple sensors, resistance thermometer sensors, infrared body thermometers, temperature strips, digital heat sensors, infrared wave sensors, and basal thermometers.

10. The electronic temperature measuring device of claim 9, wherein the thermistor sensors are attached directly to a lower extremity of a subject.

11. The electronic temperature measuring device of claim 1, wherein the power supply is a battery.

12. The electronic temperature measuring device of claim 11, wherein the battery is a rechargeable battery.

13. A method of preventing tissue damage in a lower extremity of a subject in need thereof comprising:
providing the electronic temperature measuring device of claim 1,
instructing the subject in need thereof to take a plurality of temperature readings on at least one foot of the subject in need thereof with the electronic temperature measuring device of claim 1, and
instructing a person to take remedial action based on the plurality of temperature readings.

14. The method of claim 13, further comprising processing a first data set of collected from the plurality of temperature sensors, and processing a second data set collected from the plurality of temperature sensors.

15. The method of claim 14, further comprising analyzing the first and second data sets by comparison to a baseline temperature reading.

16. The method of claim 14, further comprising storing the first and second data sets.

17. The method of claim 14, further comprising transferring the first and second data sets to a separate computing device.

18. A method of externally measuring the temperature of a lower extremity of a subject in need thereof comprising the steps of providing the electronic temperature measuring device of claim 1, and instructing a person to take a plurality of temperature readings on at least one foot of the subject in need thereof with the electronic temperature measuring device of claim 1.

19. The method of claim 18, wherein the person is selected from the group consisting of a medical professional, a caregiver, and the subject in need thereof.

20. The method of claim 18, further comprising a step of taking the plurality of temperature readings on at least one foot of the subject in need thereof with the electronic temperature measuring device of claim 1.
